(19) United States
(12) Patent Application Publication
(10) Pub. No.: US 2010/0145173 A1
(43) Pub. Date: Jun. 10, 2010

(54) SYSTEM AND METHOD FOR CREATING A PERSONALIZED TOOL PREDICTING A TIME COURSE OF BLOOD GLUCOSE AFFECT IN DIABETES MELLITUS

(76) Inventors: Clifton A. Alferness, Port Orchard, WA (US); Gust H. Bardy, Carnation, WA (US)

Correspondence Address: CASCADIA INTELLECTUAL PROPERTY, 500 UNION STREET, SUITE 1005, SEATTLE, WA, 98101, US

(21) Appl. No.: 12/030,071

(22) Filed: Feb. 12, 2008

(51) Int. Cl. A61B 5/00

(52) U.S. Cl. 600/365

(57) ABSTRACT

A system and method for establishing a tool of blood glucose change for diabetes mellitus management in an individual patient is provided. Factors specific to a diabetic patient are determined. An insulin sensitivity for an insulin preparation for treatment of diabetes mellitus is identified. A carbohydrate sensitivity for a known quantity of carbohydrate is identified, which is measured postprandial after a fixed time period. A management tool for the diabetic patient is generated. A time course for a dose of the insulin preparation with an amplitude of change proportioned to the insulin sensitivity is mapped. A time course for an amount of carbohydrate with an amplitude of change proportioned to the carbohydrate sensitivity is mapped. The management tool is calibrated by aggregating feedback from testing of blood glucose into at least one of the insulin and the carbohydrate sensitivities.

### FIELD

This application relates in general to diabetes mellitus management and, in particular, to a system and method for creating a personalized tool predicting a time course of blood glucose affect in diabetes mellitus.

### BACKGROUND

Diabetes mellitus, or simply, “diabetes,” is an incurable chronic disease. Type 1 diabetes is caused by the destruction of pancreatic beta cells in the Islets of Langerhans through autoimmune attack. Type 2 diabetes is due to defective insulin secretion, insulin resistance, or reduced insulin sensitivity. Gestational diabetes first appears during pregnancy and generally resolves after childbirth, absent preexisting weak pancreatic function. Less common forms of diabetes include thiazide-induced diabetes, and diabetes caused by chronic pancreatitis, tumors, hemochromatosis, steroids, Cushing's disease, and acromegaly.

Diabetes exacts a significant cost. In the United States, the annual healthcare costs of diabetes exceeds $200 billion. Additionally, the personal toll from diabetes is wide-ranging, impacting every patient's health and quality of life, as well as affecting the lives of the people around them. The unceasing demands of diabetes management can leave a patient feeling a loss of personal freedom, yet better control over diabetes lowers the risk of acute and chronic complications.

Type 1 diabetes can only be treated by taking insulin and making permanent lifestyle adjustments. Blood glucose and proper insulin dosing requires every Type 1 diabetic to play an active role in their own self-care. Whereas well-controlled Type 2 diabetics see relatively constrained rises and dips in blood glucose, Type 1 diabetics frequently experience wide fluctuations, known as lability or brittleness. Thus, the timing and dosing of insulin and patient-related factors, such as meals, exercise, and physiological condition, make effective blood glucose management a delicate balancing act between the prevention of hyperglycemia, or high blood glucose, and the frequent and serious consequences of hypoglycemia, or very low blood glucose, from over-aggressive or incorrect insulin dosing, which can lead to abrupt loss of consciousness.

In contrast, Type 2 diabetes is a progressive disease that requires increasing care as insulin resistance increases and insulin secretion diminishes. Initially, Type 2 diabetes can be managed through changes in physical activity, diet, and weight loss, which may temporarily restore normal insulin sensitivity. However, as insulin production becomes impaired, antidiabetic medications may be necessary to increase insulin production, decrease insulin resistance, and help regulate inappropriate hepatic glucose release. Eventually, insulin therapy will become necessary as insulin production ceases entirely.

Blood glucose management for both Type 1 and Type 2 diabetes is open loop. Devices for automatically dosing insulin based on real time blood glucose are not yet available. Instead, blood glucose management requires regular self-testing using test strips and a blood glucose meter. Self-testing is normally supplemented with glycated hemoglobin (HbAlc) or other in-clinic testing, which are performed every three to six months to evaluate long-term control. HbAlc tends to weigh recent blood glucose levels more heavily and reflects near term bias. As the time between clinic visits increases, physician interpretation of blood glucose testing results becomes less timely and, therefore, less effective.

Timely and effective diabetes management is particularly necessary to Type 1 diabetics with labile profiles and for Type 2 diabetics on insulin therapy, as time-sensitive adjustments to insulin dosing can help mitigate wide blood glucose fluctuations and their dangerous sequelae. Insulin sensitivity varies by patient. Most diabetics develop an intuition over their own sensitivity and learn to counterbalance the effects of an insulin dosing regimen. Unfortunately, well-intentioned insulin dosing is meaningless if the patient forgets to take his insulin.

Existing approaches to diabetes management still rely on physician decision-making. For instance, U.S. Pat. No. 6,168,563, to Brown, discloses a healthcare maintenance system based on a hand-held device. Healthcare data, such as blood glucose, can be uploaded on to a program cartridge for healthcare professional analysis at a centralized location. Healthcare data can also be directly obtained from external monitoring devices, including blood glucose monitors. At the centralized location, blood glucose test results can be matched with quantitative information on medication, meals, or other factors, such as exercise. Changes in medication dosage or modification to the patient's monitoring schedule can be electronically sent back to the patient. However, decision making in respect of an insulin treatment regimen through interpretation of uploaded healthcare data remains an offline process, discretionary to and within the sole control and timing of the remote healthcare professional.

Similarly, U.S. Pat. No. 6,024,699, to Surwit et al. (“Surwit”), discloses monitoring, diagnosing, prioritizing, and treating medical conditions of a plurality of remotely located patients. Each patient uses a patient monitoring system that includes medicine dosage algorithms, which use stored patient data to generate medicine dosage recommendations for the patient. A physician can modify the medicine dosage algorithms, medicine doses, and fixed or contingent self-monitoring schedules, including blood glucose monitoring through a central data processing system. In particular, diabetes patients can upload their data to the central data processing system, which will detect any trends or problems. If a problem is detected, a revised insulin dosing algorithm, insulin dosage, or self-monitoring schedule can be downloaded to their patient monitoring system. However, such modifications and revisions remain within the sole discretion and timing of a physician, who acts remotely via the central data processing system.

Notwithstanding, for the diabetic patient, guidance on what course of corrective action, either food ingestion or insulin dosing, must often be made immediately to avoid short term consequences, such as hypoglycemia. Therefore, there is a need for Type 1 and Type 2 diabetes management assistance capable of adapting a regimen to on-going patient conditions in a localized and time-responsive fashion. Preferably, such assistance would be patient-operable and integrate blood glucose self-testing and other monitoring data sources.

### SUMMARY

A system and method for modeling management of Type 1 or Type 2 diabetes mellitus on an individualized and continually fine-tunable basis is provided. An automated diabetes management tool is established by using the insulin, antidiabetic and oral medication, and carbohydrate sensitivities of a diabetic as a reference starting point. Population-based insulin and antidiabetic and oral medication activity curve data can be scaled to reflect the diabetic's personal sensitivities. A carbohydrate sensitivity can be determined through consumption of a standardized, timed test meal. A digestive response curve can be generated from the carbohydrate sensitivity by proportioning a time course curve based on postprandial blood glucose data, such as glycemic index. The personal insulin and antidiabetic and oral medication activity curves and digestive response curve form a personalized and automated diabetes management tool.

One embodiment provides a system and method for creating, a personalized tool predicting a time course of blood glucose affect in Type 1 diabetes mellitus. A substance is selected whose introduction into a diabetic patient triggers a physiological effect relative to the diabetic patient's blood glucose. A time course and an amplitude of change over which the physiological effect is expected to occur are determined. The time course and the amplitude of change are adjusted in relation to a factor specific to the physiological effect on the diabetic patient. The physiological effect is mapped on a curve with the time course and the amplitude of change mapped as a function of a quantity of the substance.

A further embodiment provides a system and method for generating a personalized diabetes management tool for Type 1 diabetes mellitus. An insulin activity curve for a patient population for an insulin preparation for Type 1 diabetes mellitus treatment is identified. A personal insulin activity model for the patient is generated. An insulin sensitivity is determined by taking a derivative of the rate of change of blood glucose over time for the insulin preparation. An insulin sensitivity coefficient for the insulin preparation for a patient of Type 1 diabetes mellitus is established. The insulin sensitivity coefficient is applied to the patient population insulin activity curve over a duration of action of the insulin preparation.

A still further embodiment provides a system and method for establishing a tool of blood glucose change for Type 1 diabetes mellitus management in an individual patient. Factors specific to a diabetic patient are determined. An insulin sensitivity for an insulin preparation for treatment of Type 1 diabetes mellitus is identified. A carbohydrate sensitivity for a known quantity of carbohydrate is identified, which is measured postprandial after a fixed time period. A management tool for the diabetic patient is generated. A time course for a dose of the insulin preparation is mapped with an amplitude of change proportioned to the insulin sensitivity. A time course for an amount of carbohydrate is mapped with an amplitude of change proportioned to the carbohydrate sensitivity. The management tool is calibrated by aggregating feedback from testing of blood glucose into at least one of the insulin and the carbohydrate sensitivities.

The personal predictive management tool provides Type 1 diabetics with a new-found sense of personal freedom and safety by integrating the vagaries of daily blood glucose control into a holistic representation that can be continually re-evaluated and calibrated to keep pace with the unpredictable nature of daily life. The approach described herein closely approximates what a normal pancreas does by interactively guiding the individual diabetic under consideration and, over time, learning how the patient can be understood and advised.

Still other embodiments of the present invention will become readily apparent to those skilled in the art from the following detailed description, wherein are described embodiments by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a functional block diagram showing, by way of example, a prior art diabetes management cycle for a Type 1 diabetic.

FIG. 2 is a functional block diagram showing, by way of example, an automated diabetes management cycle for a Type 1 diabetic, in accordance with one embodiment.

FIGS. 3A-C are functional block diagrams showing, by way of example, prior art diabetes management cycles for a Type 2 diabetic.

FIGS. 4A-C are functional block diagrams showing, by way of example, automated diabetes management cycles for a Type 2 diabetic, in accordance with one embodiment.

FIG. 5 is a process flow diagram showing personalized Type 1 and Type 2 diabetes mellitus modeling.

FIG. 6 is a diagram showing, by way of example, a screen shot of a graphical user interface for establishing a personalized diabetes management tool for Type 1 and Type 2 diabetes mellitus.

FIG. 7 is a diagram showing, by way of example, a screen shot of a graphical user interface for specifying insulin preparation type for use in the graphical user interface of FIG. 6

FIG. 8 is a diagram showing, by way of example, a screen shot of a graphical user interface for specifying other medications for use in the graphical user interface of FIG. 6.

FIG. 9 is a diagram showing, by way of example, a screen shot of a graphical user interface for selecting food combinations for use in the graphical user interface of FIG. 6.

FIG. 10 is a process flow diagram showing a method for establishing a personalized diabetes management tool for Type 1 and Type 2 diabetes mellitus, in accordance with one embodiment.

FIG. 11 is a process flow diagram showing a routine for establishing an insulin activity curve for use with the method of FIG. 10.

FIG. 12 is a process flow diagram showing a routine for calibrating an insulin activity curve for use with the method of FIG. 10.

FIG. 13 is a process flow diagram showing a routine for establishing an antidiabetic and oral medication activity curve for use with the method of FIG. 10.

FIG. 14 is a process flow diagram showing a routine for calibrating an antidiabetic and oral medication activity curve for use with the method of FIG. 10.

FIG. 15 is a graph showing, by way of example, an insulin activity curve for lispro, an insulin analog.

FIG. 16 is a diagram showing, by way of example, a screen shot of a personal insulin activity curve for display in the graphical user interface of FIG. 6.

FIG. 17 is a process flow diagram showing a routine for establishing a digestive response curve for use with the method of FIG. 10.

FIG. 18 is a graph showing, by way of example, a personal digestive response curve for a standardized meal.

FIG. 19 is a process flow diagram showing, by way of example, characteristics affecting diabetes management.

FIG. 20 is a process flow diagram showing, by way of example, factors bearing on personal predictive diabetes management.

FIG. 21 is a block diagram showing for a system for generating a personalized diabetes management tool for Type 1 diabetes mellitus, in accordance with one embodiment.

### DETAILED DESCRIPTION

### Diabetes Management Cycles

Both Type 1 and Type 2 diabetes are diseases that require continuous and consistent glucose management. Poorly controlled diabetes affects both quality of life and longevity, which can be dramatically curtailed due to avoidable chronic complications. Conversely, acute conditions will occur with even well-managed patients, although proper management helps to significantly lower the likelihood.

Type 1 Diabetes

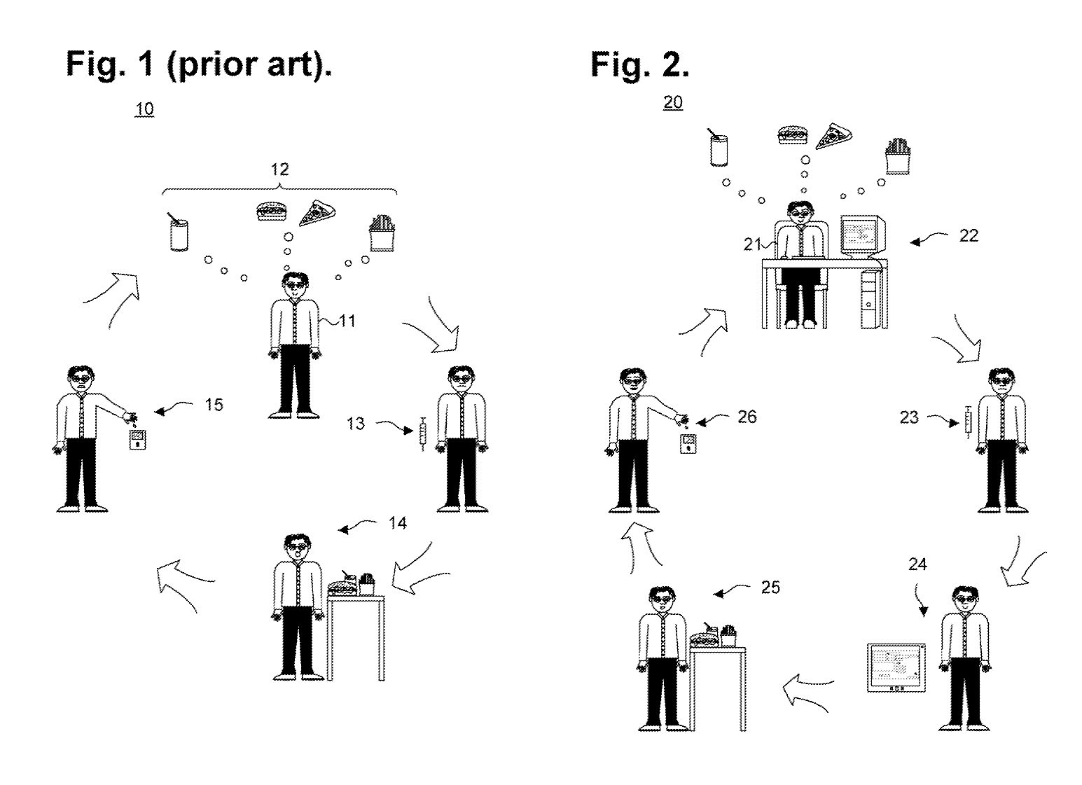

The principal cause of Type 1 diabetes is a T-cell mediated autoimmune attack on the beta cells of the islets of Langerhans of the pancreas. No known preventative measures exist. Type 1 diabetes management is a continual cycle that is repeated on a daily basis. FIG. 1 is a functional block diagram showing, by way of example, a prior art diabetes management cycle 10 for a Type 1 diabetic. Fundamentally, Type 1 diabetes management centers oil the timing and content of meals and the timing and dosing of insulin, although other factors, such as physical activity and exercise, overall physical well-being, illness, and stress, can influence the course of management.

Currently, Type 1 diabetes can only be treated through insulin therapy, which is normally combined with adjustments to patient lifestyle, including diet and exercise. As a result, a Type 1 diabetic patient 11 learns to plan and time his daily meals (step 12) to estimate an expected rise in blood glucose and to determine appropriate doses of insulin to counteract the expected rise.

Generally, a Type 1 diabetic administers insulin prior to actually consuming any food (step 13). A post-Meal increase in blood glucose is normal, but the insulin in intended to bring blood glucose back down to a reasonable range within two to four hours. A Type 1 diabetic determines the insulin units needed to counteract an expected post-meal rise in blood glucose and times his insulin to counteract the affect of the meal (step 14). Ideally, a Type 1 diabetic's average blood glucose should be in the range of 20-120 mg/dL, although a range of 140-150 mg/dL is often used to prevent potentially life-threatening hypoglycemic events. In effect, long-term management of blood glucose levels is shortchanged to prevent the more pressing short-term consequences of hypoglycemia.

Physicians encourage each Type 1 diabetic to regularly self-test his blood glucose (step 15) to enable better compensation for patient-specific sensitivities to both food and insulin. A patient 11 places a drop of blood on a test strip coated with a glucose oxidase or hexokinase enzyme, which is read by a glucose monitor. Blood glucose is normally tested daily, although stricter control regimens may require more frequent testing.

Patient logs document the interaction of food, insulin, and patient sensitivities. Physician review normally only occurs during clinic visits, or when otherwise necessary. Detailed context is lost, unless the patient comprehensively records exacting descriptions of all food components consumed and their manner of preparation, precise times between insulin dosing and completion of a meal, physiological factors, such as mood or wellness, and similar data. The physician must be willing to study a patient log in corresponding detail. However, neither detailed patient documentation nor close physician review are practical in terms of time, effort, and cost for every Type 1 diabetic patient.

The accuracy and timeliness of a Type 1 diabetes management regimen can be improved by automating the predictive aspects of glycemic control. FIG. 2 is a functional block diagram showing, by way of example, an automated diabetes management cycle 20 for a Type 1 diabetic, in accordance with one embodiment. Automation is introduced to move the management cycle significantly closer to a closed loop arrangement. Control is streamlined and steps that remain to be performed by a patient manually are minimized, or even eliminated, which make such steps less apt to be forgotten or missed.

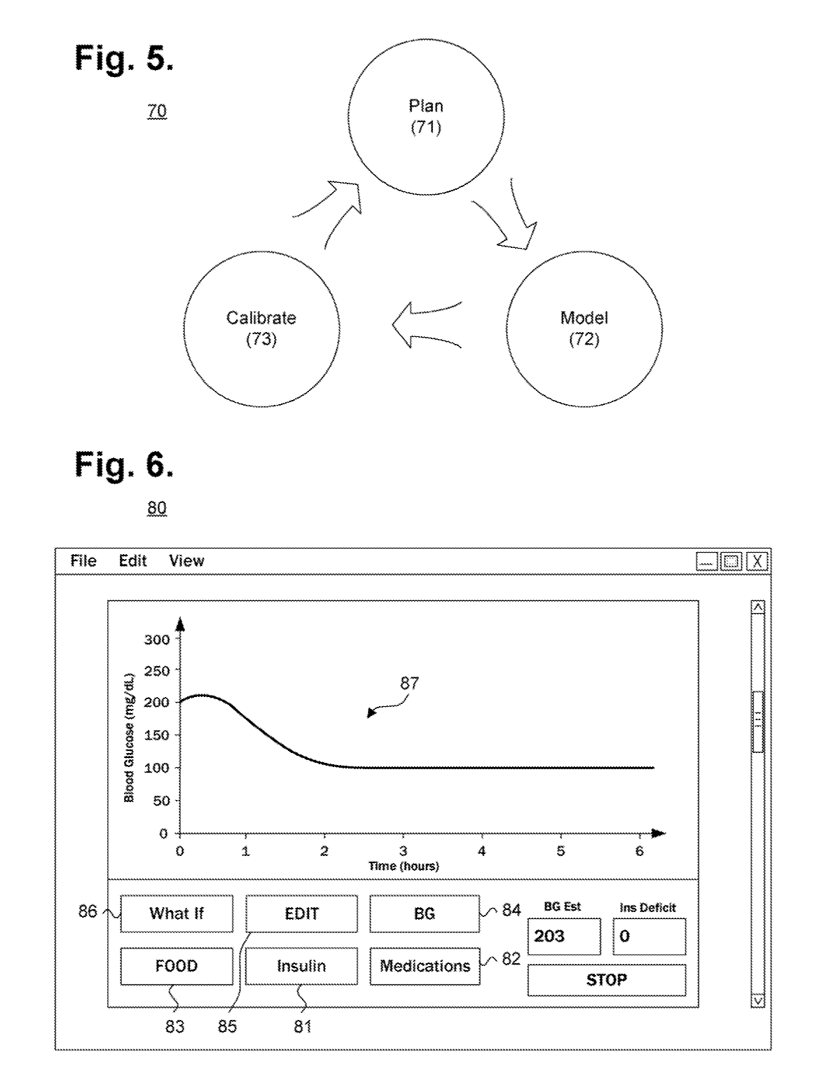

An automated diabetes management tool applies heuristics to model and calibrate a personalized diabetes control regimen for a Type 1 diabetic patient 21 (step 22), as further described below beginning with reference to FIG. 5. Through the management tool, the patient 21 can plan and time insulin dosing and meals (steps 23 and 25, respectively) more accurately than possible through conventional means, including exchange lists and carbohydrate counting. Dynamically tracked blood glucose predictions (step 24) can also be provided and self-testing results (step 26) can be provided directly into the management tool for time-responsive integration and evaluation. In a further embodiment, emergent glucose self-testing, such as interstitial glucose testing, supplements manual self-testing or, where sufficiently reliable, replaces manual self-testing to achieve a fully closed loop system when combined with insulin pump therapy. Other management tool aspects are possible.

Type 2 Diabetes

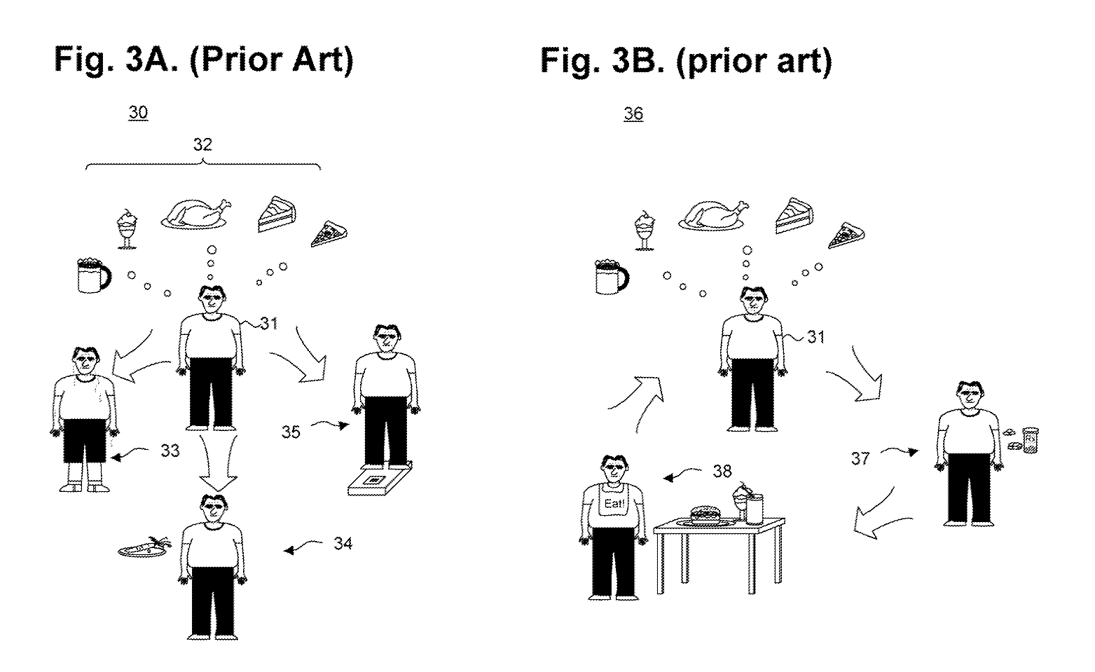

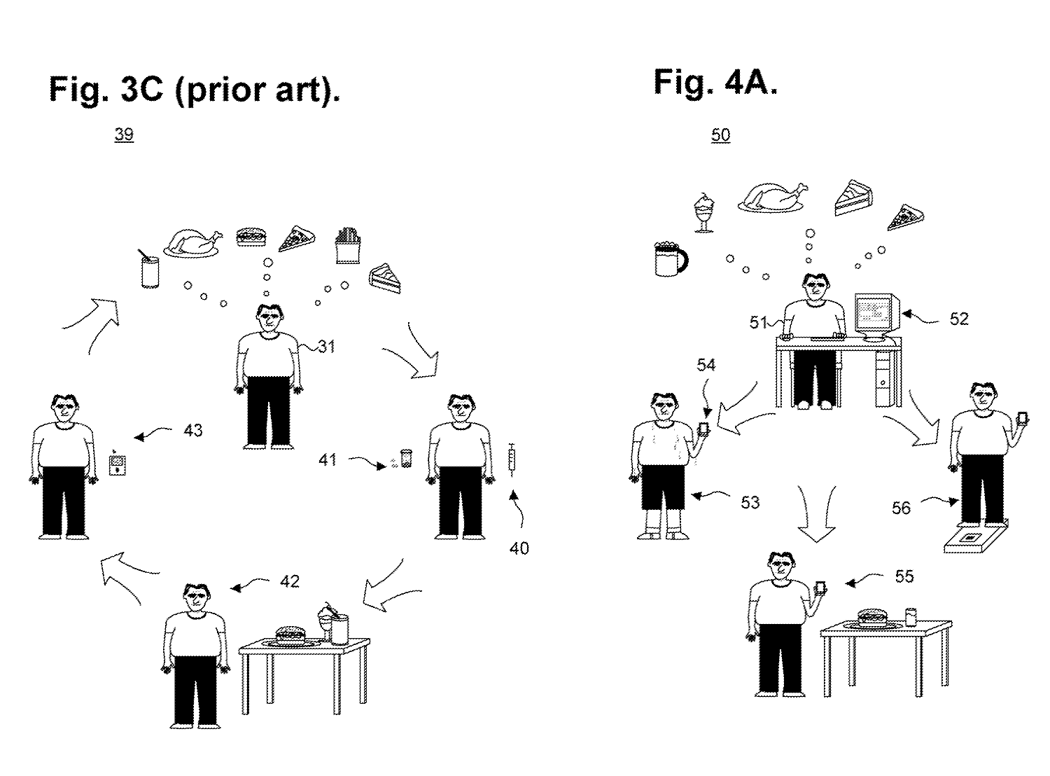

Type 2 diabetes is due to defective insulin secretion, insulin resistance, or reduced insulin sensitivity. No known preventative measures exist, either, but strong correlations to obesity and genetic predisposition have been observed. Like Type 1 diabetes, Type 2 diabetes management is a continual cycle that is repeated on a daily basis, but the nature and amount of management changes as the disease progresses over time. FIGS. 3A-C are functional block diagrams showing, by way of example, prior art diabetes management cycles for a Type 2 diabetic. The earliest stage of Type 2 diabetes can be controlled through lifestyle changes alone, after which antidiabetic medications and ultimately insulin therapy are eventually added.

Early stage Type 2 diabetes management focuses on changes in lifestyle with emphasis on basic glycemic control. Referring first to FIG. 3A, a typical patient 31 is often obese, although obesity is but one indicator of Type 2 diabetes, which further includes genetic predisposition and mutation of amylin genes. Diet 32 often plays a significant role. As a result, the patient 31 is urged to exercise, for instance, by taking a brisk 45-minute walk several times a week, and to increase his physical activity (step 33) through a combination of aerobic and resistance training. In addition, the patient 31 is educated on following a healthy diet (step 34) to decrease his weight (step 35), as the level of insulin resistance proportionately grows with increase in body fat, particularly metabolically active visceral fat.

Effective early stage Type 2 diabetes control can temporarily restore normal insulin sensitivity, although the predisposition for insulin resistance remains. Referring next to FIG. 3B, antidiabetic and oral medications are generally prescribed (step 37) as insulin production becomes impaired, yet partial pancreatic function remains. Most commonly, beguanide metformin and sulfonylureas are prescribed to respectively help regulate inappropriate hepatic glucose release and stimulate insulin production. Thiazolidinediones may also be prescribed, which decrease insulin resistance. Lifestyle changes (step 38) in exercise, diet, and weight loss continue.

In the last stage, pancreatic function ceases altogether, which necessitates commencement of insulin therapy. Referring to FIG. 3C, insulin is administered transvenously (step 40). Insulin therapy is performed in a manner similar to a Type 1 diabetic, which includes both meal and insulin dosage planning, as described above with reference to FIG. 1. However, antidiabetic and oral medications (step 41) may also be taken, along with continued adherence to lifestyle changes (step 42). Additionally, the patient 31 is now encouraged to self-test his blood glucose (step 43).

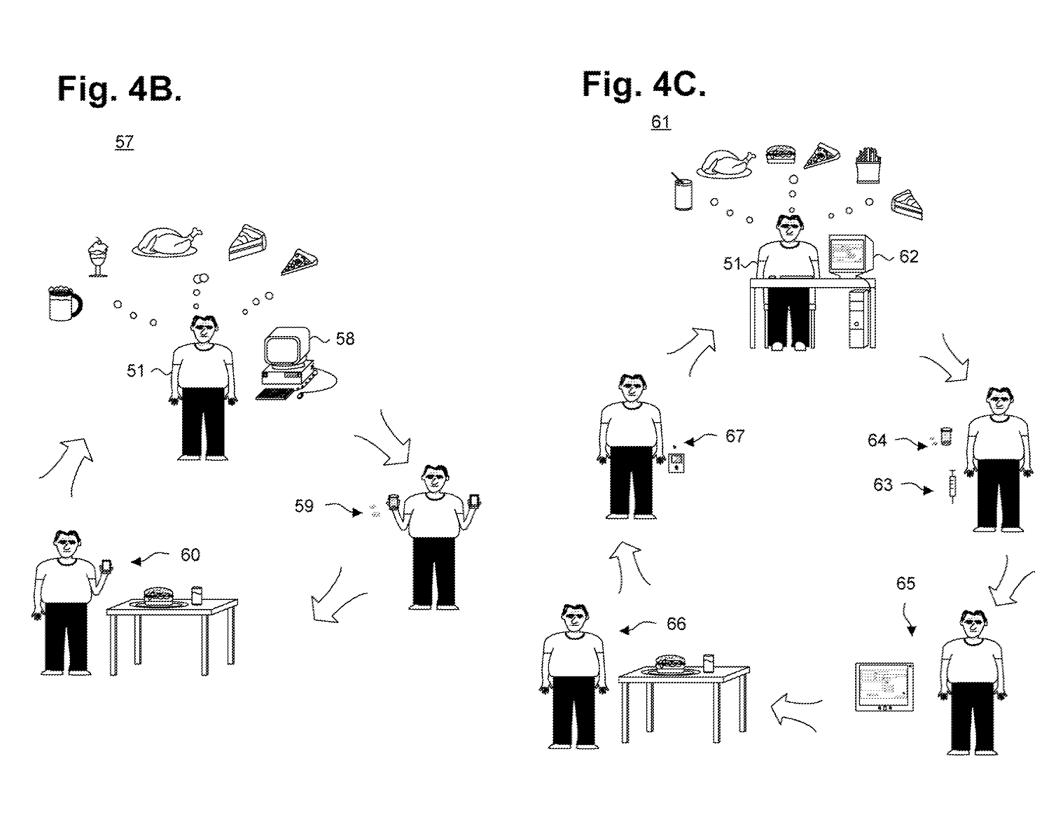

Many aspects of Type 2 diabetes management can also be automated. FIGS. 4A-C are functional block diagrams showing, by way of example, automated diabetes management cycles for a Type 2 diabetic, in accordance with one embodiment. In a manner similar to Type 1, automation is introduced to increase the accuracy and timeliness of blood glucose control and logging, and to minimize or eliminate steps performed by a patient manually.

Type 2 diabetes is a progressively debilitating disorder and quality of life can best be preserved by seeding diabetes awareness from the earliest stages of the disease. Referring first to FIG. 4A, a Type 2 diabetic patient 51 faces making changes to his lifestyle through physical activity (step 53), diet (step 55), and weight (step 56). Hopefully, the changes are permanent, but a diabetic 51 may lose sight of their importance, either through indifference or by temporary restoration of insulin sensitivity. Consequently, during early stage Type 2 diabetes, an automated diabetes management tool can be used to assist the patient 51 in planning his diet and physical activities (step 52), and in tracking his progress (step 54) for subsequent review and analysis, as further described below beginning with reference to FIG. 5.

As insulin resistance increases and pancreatic function decreases, antidiabetic and oral medications become increasingly important. Referring next to FIG. 4B, the types and timing of medications required will depend upon the patient's physiology and physical tolerance. Moreover, medication may be necessary at different times of the day and in different combinations. In addition to planning (step 58) and tracking (step 60) functions, the management tool can also provides dosing instructions and reminders (step 59) to guide the patient 51 in therapy compliance.

End-stage Type 2 diabetes introduces insulin therapy. Referring finally to FIG. 4C, insulin requires conscious planning (step 62) and conscientious dosing (step 63), both in appropriate amount and at the correct time with respect to anticipated meals to effectively lower the blood sugar and to prevent the negative consequences of hypoglycemia. The management tool applies planning (step 62) and tracking (step 66) functions similar in form to the methodology of Type 1 diabetes, but further includes administration of antidiabetic and oral medications (step 64). In addition, the management tool can provide dynamic blood glucose prediction (step 65) and blood glucose self-testing integration (step 67). In a further embodiment, interstitial glucose testing and similar diagnostics supplement or replace manual self-testing, which provide a fully closed loop system when combined with a wireless insulin pump. Other management tool aspects are possible.

### Automated Management of Type 1 and Type 2 Diabetes

The diabetic patient is himself the best resource available to manage diabetes. Meals, insulin dosing, and changes in personal well being, as well as departures from such plans, are best known to the patient, who alone is ultimately responsible for adherence to a management regimen. FIG. 5 is a process flow diagram showing personalized Type 1 and Type 2 diabetes mellitus modeling 70. The method is performed as a series of process steps or operations executed by one or more patient-operable devices, including personal computers, personal digital assistants, programmable mobile telephones, intelligent digital media players, or other programmable devices, that are working individually or collaboratively to apply a personal predictive management tool.

Modeling involves projecting the glycemic effect of planned meals in light of insulin dosing, if applicable, and antidiabetic and oral medications (primarily Type 2). Meal planning is particularly important to Type 1 and end-stage Type 2 diabetics, where the content and timing of meals greatly impacts blood glucose and must be closely controlled by dosed insulin to compensate for the lack of naturally-produced insulin. Dietary management is less crucial to non-end-stage Type 2 diabetics, who still retain limited natural insulin production. Nevertheless, proper diet can aid with weight control and hepatic glucose release. For all Type 1 and Type 2 diabetics, the management tool performs dietary planning (step 71), which primarily involves determining the glycemic effect of food based on a standardized meal. In a further embodiment, planning also includes projecting the affect of exercise or physical activities that are likely to require appreciable caloric expenditure. Other planning aspects are possible.

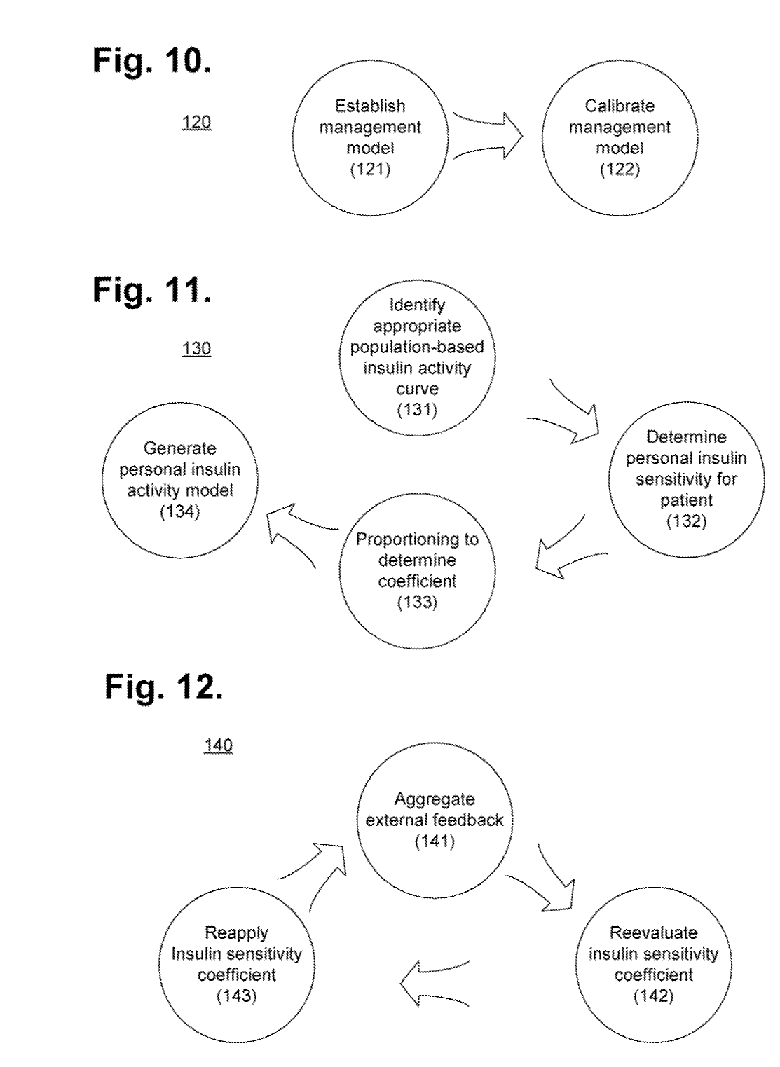

Once each planned meal is known, the management tool can model the time courses and amplitudes of change for the meal, dosed insulin, and antidiabetic and oral medication (primarily Type 2) (step 72). Additionally, the management tool can be calibrated as necessary to adjust to self-testing and data recorded by the patient (step 73), as further described below beginning with reference to FIG. 10. Other modeling and calibration aspects are possible.

In a further embodiment, the patient can combine different food types and quantities and perform “What If” scenarios as an aid to blood glucose management. The physiological effects on blood glucose of specific food and beverage, both individually and in combination, are modeled, taking into account differences in digestive motility and other factors, such as described in commonly-assigned U.S. patent application, entitled “System And Method For Actively Managing Type 1 Diabetes Mellitus On A Personalized Basis,” Ser. No. ______, pending; U.S. patent application, entitled “System and Method for Managing Type 1 Diabetes Mellitus Through a Personal Predictive Management Tool,” Ser. No. ______, pending, U.S. patent application, entitled “System And Method For Actively Managing Type 2 Diabetes Mellitus On A Personalized Basis,” Ser. No. ______, pending; U.S. patent application, entitled “System and Method for Managing Type 2 Diabetes Mellitus Through a Personal Predictive Management Tool,” Ser. No. ______, pending, the disclosures of which are incorporated by reference. Tile modeling is based on an individual patient's personal dietary tastes and preferences and the blood glucose rises that ensue following consumption, as Well as capturing the synergies and interactions of various food preparations and combinations.

### Graphical User Interface

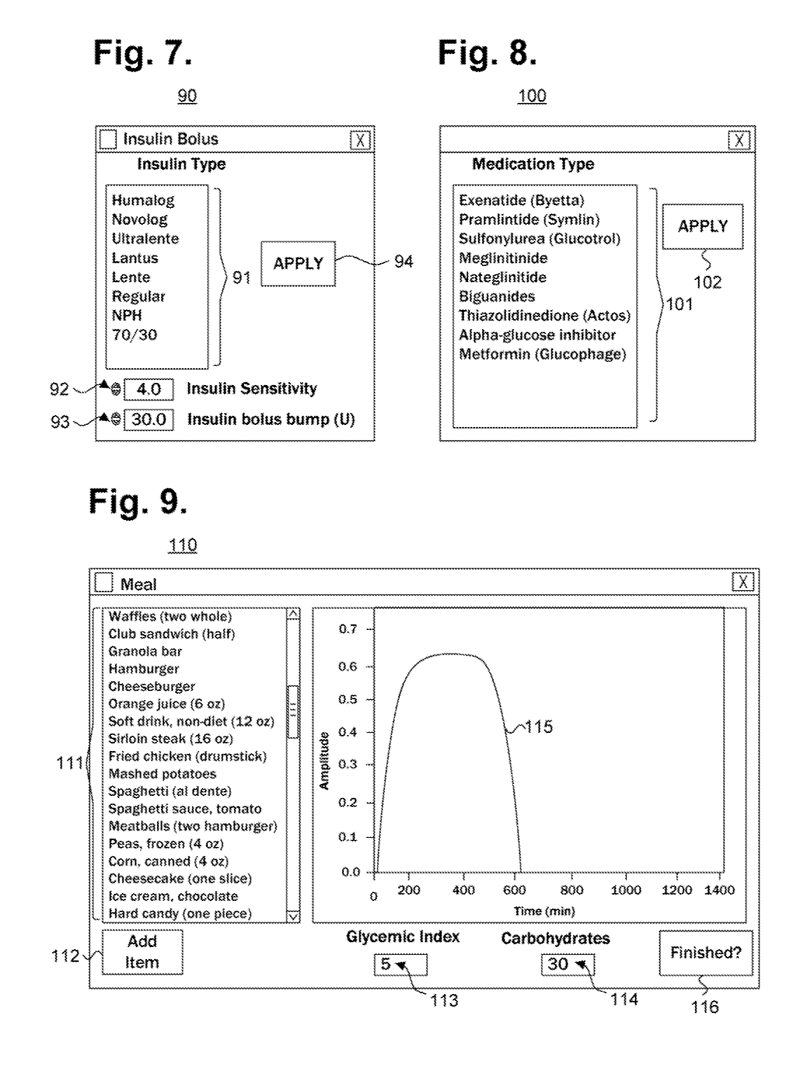

Personalized Type 1 and Type 2 diabetes mellitus modeling can be provided through a patient-operable interface through which planning and calibration can be performed. FIG. 6 is a diagram showing, by way of example, a screen shot of a graphical user interface 80 for establishing a personalized diabetes management tool for Type 1 and Type 2 diabetes mellitus, in accordance with one embodiment. The user interface 80 provides logical controls that accept patient inputs and display elements that present information to the patient. The user interface 80 can be used for both Type 1 and Type 2 diabetes management, although the applicability of particular logical controls, screens, and menus will depend upon the type and stage of the patient's diabetes. The logical controls include buttons, or other forms of option selection, to access further screens and menus to specify an insulin bolus 81 (“Insulin”), as further described below with reference to FIG. 7; specify other antidiabetic and oral medications 82 (“Medications”) (primarily Type 2), as further described below with reference to FIG. 8; plan meals 83 (“FOOD”), as further described below with reference to FIG. 9; enter a measured blood glucose reading 84 (“BG”); edit information 85 (“EDIT”); and speculate on what would happen if some combination of food, insulin, or antidiabetic or oral medication (primarily Type 2) were taken 86 (“What If”). Further logical control and display elements are possible.

To assist the patient with planning, a graphical display provides a forecast curve 87, which predicts combined insulin dosing, antidiabetic and oral medication administration (primarily Type 2), and postprandial blood glucose. The x-axis represents time in hours and the y-axis represents the blood glucose level measured in mg/dL. Modeling estimates the timing and amplitude of change in the patient's blood glucose in response to the introduction of a substance, whether food, physiological state, or drug, that triggers a physiological effect in blood glucose. Generally, actions, such as insulin dosing, medication administration, exercise, and food consumption cause a measureable physiological effect, although other substances and events can influence blood glucose. The time courses and amplitudes of change are adjusted, as appropriate, to compensate for patient-specific factors, such as level of sensitivity or resistance to insulin, insulin secretion impairment, carbohydrate sensitivity, and physiological reaction to medications. Other patient-specific factors, like exercise or supervening illness, may also alter the time courses and amplitudes of blood glucose.

In one embodiment, the user interface 80 and related components are implemented using a data flow programming language provided through the LabVIEW development platform and environment, licensed by National Instruments Corporation, Austin, Tex. although other types and forms of programming languages, including functional languages, could be employed. The specific option menus will now be discussed.

Insulin Selection

When insulin therapy is applicable, such as for a Type 1 or end-stage Type 2 diabetic, a patient needs to identify both the type and amount of insulin preparation used and his sensitivity to allow the management tool to generate an insulin response curve. Insulin preparation types are identified by source, formulation, concentration, and brand name, and are generally grouped based on duration of action. FIG. 7 is a diagram showing, by way of example, a screen shot of a graphical user interface 90 for specifying insulin preparation type for use in the graphical user interface 80 of FIG. 6. Different types of insulin preparation 91 can be selected and, for ease of use and convenience, are identified by brand name or formulation. Insulin preparations include short-acting insulins, such as lispro or Regular, are used to cover a meal and are frequently administered by insulin pump due to the short time of onset. Intermediate-acting insulins, such as NPH (Neutral Protamine Hagedorn), have 12-18 hour durations, which peak at six to eight hours. Finally, long-acting insulins, such as Ultralente, have 32-36 hour durations to provide a steady flow of insulin. Long-acting insulins are generally supplemented with short-acting insulins taken just before meals. Other types of insulin preparations include insulin glargine, insulin detemir, and insulin preparation mixes, such as “70/30,” which is a premixed insulin preparation containing 70% NPH insulin and 30% Regular insulin. In addition, insulin sensitivity 92 and insulin bolus “bump” 93, that is, a single dosing, such as for meal coverage, is specified, before being factored into the tool upon pressing of the “APPLY” button 94. Further logical control and display elements are possible.

Other Medication Selection (Primarily Type 2)

Type 2 diabetics generally start with antidiabetic and oral medications and only later progress to insulin therapy as insulin production ceases. However, Type 1 diabetics also may receive medications in addition to insulin. Each medication should also be identified to allow the management tool to project any effect on glycemic activity. FIG. 8 is a diagram showing, by way of example, a screen shot of a graphical user interface 100 for specifying other medications for use in the graphical user interface of FIG. 6. Different medications 101 can be selected and, for ease of use and convenience, can be identified by generic name, brand name, or formulation. As appropriate, the therapeutic effects, particularly as relating to blood glucose level, and drug interactions of each medication can be factored into the tool upon pressing of the “APPLY” button 102. For example, pramlintide acetate, offered under the brand name Symlin, is prescribed to both Type 1 and Type 2 diabetics help lower postprandial blood glucose during the three hours following a meal. Consequently, the blood glucose rise is adjusted to reflect the effects of the pramlintide acetate in light of a planned meal and dosed insulin, if applicable. Further logical control and display elements are possible.

Food Selection

Unlike insulin preparation or other medications, the possible selections and combinations of food and beverage are countless and applicable, whether Type 1 or Type 2 diabetic, regardless of disease state. Moreover, how a particular food combination synergistically acts is equally variable. FIG. 9 is a diagram showing, by way of example, a screen shot of a graphical user interface 110 for selecting food combinations for use in the graphical user interface 80 of FIG. 6. Dietary management, and thence, the management tool, focuses on carbohydrates, which have the greatest affect on blood glucose. Simple sugars increase blood glucose rapidly, while complex carbohydrates, such as whole grain bread, increase blood glucose more slowly due to the time necessary to break down constituent components. Fats, whether triglyceride or cholesterol, neither raise nor lower blood glucose, but can have an indirect affect by delaying glucose uptake. Proteins are broken down into amino acids, which are then converted into glucose that will raise blood glucose levels slowly. Proteins will not generally cause a rapid blood glucose rise. Nevertheless, both fats and proteins are incorporated into the model by virtue of their empiric effect on blood glucose levels. Additionally, various combinations or preparations of medications or food can have synergistic effects that can alter blood glucose rise and timing.

In the management tool, the food choices 111 are open-ended, and one or more food item can be added to a meal by pressing, the “ADD ITEM” button 112. Glycemic effect data, such as the glycemic index 113 and carbohydrates type and content 114 for a particular food item, are also displayed. A cumulative digestive response curve 115 is generated and is mapped to run contemporaneous to the insulin activity curve, so the affect of an insulin dose can be weighed against food ingestion. The cumulative digestive response curve 115 is based on the selections made by proportionately applying the patient's carbohydrate sensitivity. For instance, the selection of a 12-ounce non-diet soft drink and a 16-ounce sirloin steak would result in a cumulative digestive response curve with an initial near term peak, which reflects the short time course and high glucose content of the soft drink, and a long term peak, which reflects the protein-delayed and significantly less-dramatic rise in blood glucose attributable to the sirloin steak. The completion of meal planning is indicated by pressing the “Finished” button 116. Further logical control and display elements are possible.

### Method

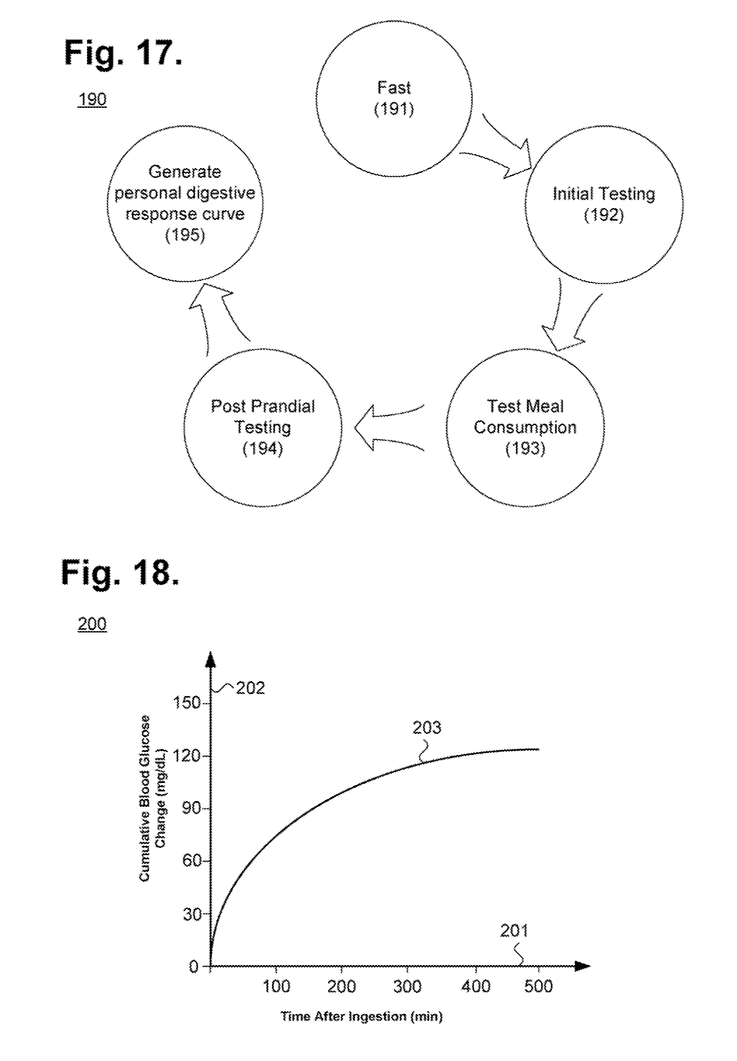

Conventional Type 1 and Type 2 diabetes management is predicated on application of population-based norms, which can serve as a starting point for personalized care. Individualized diabetes management adapts these norms to a model to meet specific patient needs and sensitivities and the model can be continually updated and fine tuned to address dynamic conditions. FIG. 10 is a process flow diagram showing a method 120 for establishing a personalized diabetes management tool for Type 1 and Type 2 diabetes mellitus, in accordance with one embodiment. The method first establishes a personal predictive management tool (operation 121). One aspect of the model is determined for insulin activity, as further described below with reference to FIG. 11, and a second aspect is determined for digestive speed and amplitude, as further described below with reference to FIG. 17. Other aspects of the management tool are possible.

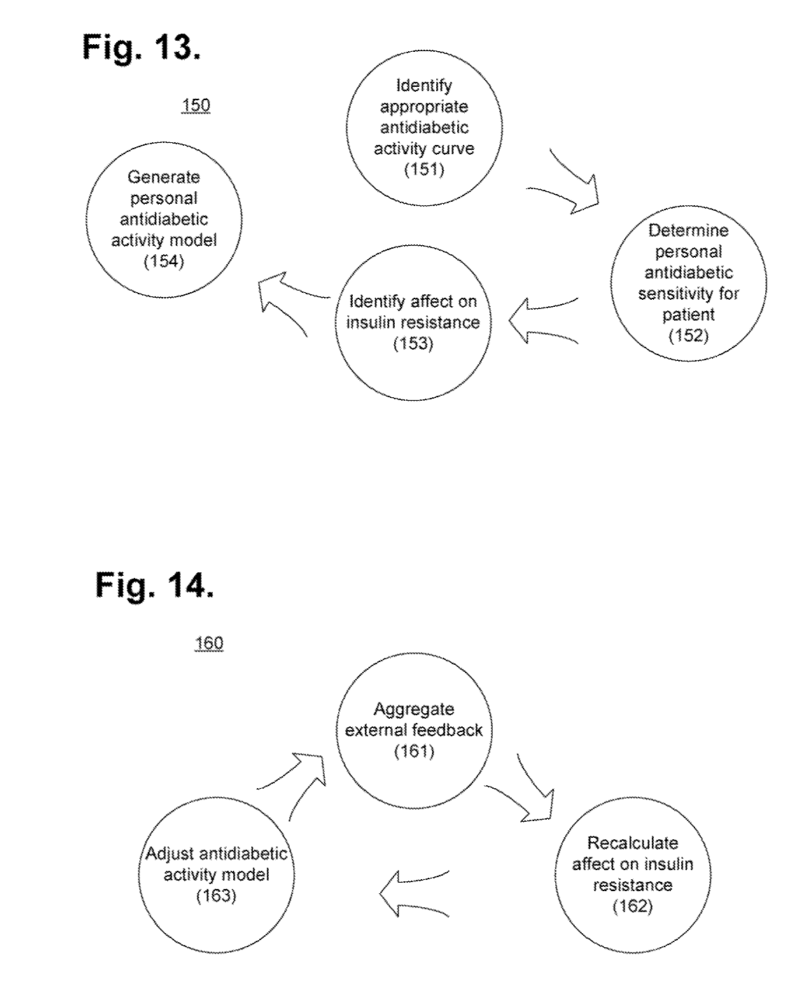

Once established, the management tool can be refined and calibrated on an on-going basis (operation 122) by integrating self-testing and other patient data sources in a localized and time-responsive fashion, as further described below respectively with reference to FIG. 13 for insulin activity. Through calibration, the management tool continually changes as more data is obtained and keeps pace with the patient over time.

### Insulin Activity Modeling

Insulin is dosed in Type 1 and end-stage Type 2 diabetics to counteract the postprandial rise in blood glucose. Insulin activity is initially modeled with an insulin activity curve for a patient population as published for a specific insulin preparation. The insulin activity curve is adapted to each specific patient by factoring individual sensitivities into the personal predictive management tool.

In a further embodiment, the management tool requires the inclusion of dosed insulin for Type 1 and end-stage Type 2 diabetics. Requiring the modeling of an activity time curve for dosed insulin is particularly important when antidiabetic or oral medications are also modeled, as the latter can skew blood glucose and cause an incomplete impression of glycemic effect if dosed insulin is omitted from the model.

Establishing an Insulin Activity Curve

The clinical pharmacologies of various types of insulin are widely available from their manufacturer. The three major manufacturers of insulin are Eli Lilly, Novo Nordisk, and Sanofi Aventis, although other manufacturers exist. Each pharmacology typically includes a time course of action based on population-based clinical studies. Time courses are provided as general guidelines, which can vary considerably in different individuals or even within the same individual depending upon activity and general health. Time courses can serve as the basis of a management tool. FIG. 11 is a process flow diagram showing a routine 130 for establishing an insulin activity curve for use with the method 120 of FIG. 10. A model is generated for a specific type of insulin preparation. Similar models for other insulin preparation types can be postulated by extension, or established individually on a case-by-case basis.

Initially, a population-based insulin activity curve that is appropriate to a particular patient is identified (operation 131). A published time course of action for the insulin preparation type can be used, which provides an established baseline for insulin activity that can be adapted to the patient. Other sources of insulin activity curves can be used, so long, as the curve accurately reflects time of onset, peak time, duration, or other essential insulin activity characteristics.

The personal level of sensitivity to the insulin preparation must also be determined for the patient (operation 132). Personal insulin sensitivity can be determined empirically, such as taking an empirically observed decrease in blood glucose for a fixed dose of the insulin preparation as the insulin sensitivity. In a further embodiment, personal insulin sensitivity can be determined by adapting interstitial glucose level for a fixed dose of the insulin preparation to blood glucose level. Other determinations of personal insulin sensitivity are possible, including clinically-derived values.

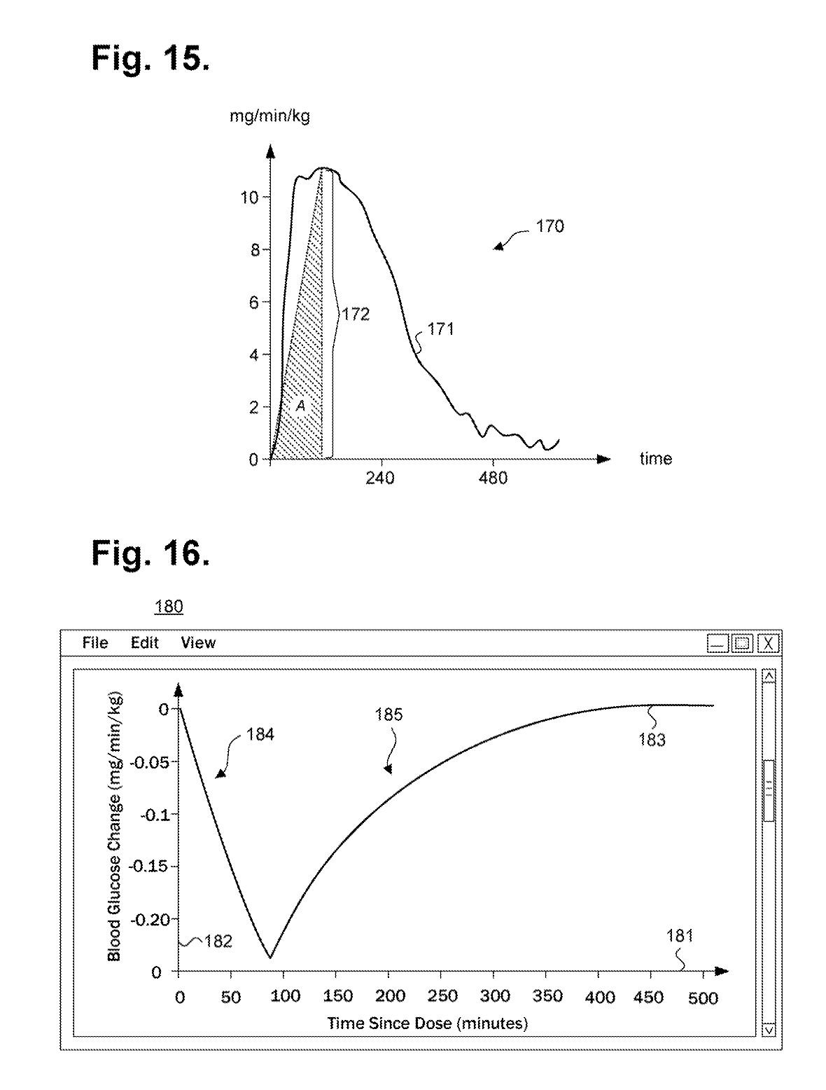

Based on the personal insulin sensitivity, an insulin sensitivity coefficient or coefficients can be found by proportioning the personal insulin sensitivity to the population-based insulin activity curve (operation 133). The coefficient can be determined through area estimation, as further described below with reference to FIG. 15. Finally, once determined, the coefficient can be applied to the population-based insulin activity curve to generate a personal insulin activity model (operation 134). An application of an insulin sensitivity coefficient is further described below with reference to FIG. 16.

Calibrating an Insulin Activity Curve

Diabetes management needs can change over time, as dietary habits, personal well being, and other factors occur in a diabetic's life. Thus, the management tool accommodates evolving conditions to remain current and continue to provide effective guidance. FIG. 13 is a process flow diagram showing a routine 150 for calibrating an insulin activity curve for use with the method 120 of FIG. 10. Calibration is a dynamic process that builds on conventional self-testing and diabetes control information ordinarily only recorded as static data for potential physician consideration.

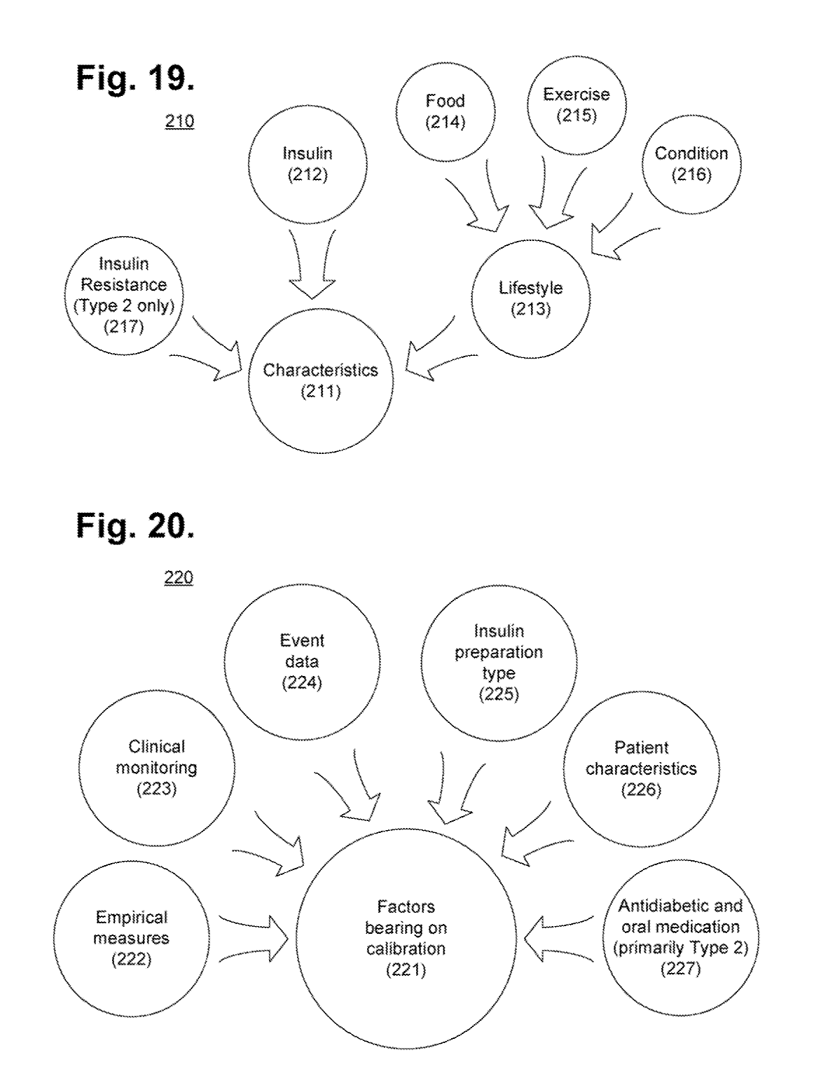

Calibration can be performed regularly, or only as needed. Various concerns can change how a management tool is characterized for a Type 1 diabetic, including factors relating to insulin, antidiabetic and oral medication, and lifestyle, as further described below with reference to FIG. 19. During each calibration, external feedback regarding the dosed insulin is aggregated into the management tool (operation 151) and applied to re-evaluate the insulin sensitivity coefficient (operation 152). The insulin sensitivity coefficient is affected only where the feedback reflects a non-nominal departure from an earlier provided sensitivity. A non-nominal departure occurs, for instance, when an observed decrease in blood glucose differs from a predicted blood glucose decrease by one or more standard deviations, although other thresholds or metrics of significance are possible. In addition, the feedback can be aggregated over several sample sets, or types of feedback, as further described below with reference to FIG. 20. A revised personal insulin activity model can then be generated (operation 153) as a reflection of current circumstances

### Antidiabetic and Oral Medication Activity Modeling (Primarily Type 2)

Antidiabetic and oral medications are prescribed only to Type 2 diabetics during the middle and final stages of the disease and are selectively used with Type 1 diabetics. The type of affect on blood glucose depends upon the drug's pharmacology and the patient's sensitivity to the drug. For example, a meglitinide stimulates the release of natural insulin, which has the affect of directly lowering blood glucose. In contrast, thiazolidiniediones increase insulin receptivity by stimulating glucose update, which indirectly lowers blood glucose, but is also dependent upon patient's insulin sensitivity. As a result, an activity curve can be generated based on population-based studies, but will ordinarily require adjustment in the management tool for each patient.

Establishing an Antidiabetic or Oral Medication Activity Curve

Like insulin, the clinical pharmacologies of the different varieties of antidiabetic and oral medications are widely available from their manufacturer and typically include a time course of action based on population-based clinical studies. In general, the population-based time courses can serve as the basis of a management tool. FIG. 13 is a process flow diagram showing a routine 150 for establishing an antidiabetic and oral medication activity curve for use with the method 120 of FIG. 10. A model is generated for each antidiabetic or oral medication identified by the patient. Similar models for other medications can be postulated by extension, or established individually on a case-by-case basis.

Initially, a population-based activity curve that is appropriate to a particular patient is identified (operation 151). A published time course of action can be used. In a further embodiment, an empirical time course of action is determined by monitoring the patient's blood glucose following dosing of the medication. Other sources of activity curves can be used, which accurately reflect time of onset, peak time, duration, or other essential activity characteristics.

The patient's level of sensitivity to the medication is also determined (operation 152), which can be found empirically. In a further embodiment, the sensitivity can be determined by adapting interstitial glucose level for a fixed dose to blood glucose level. Other determinations of insulin sensitivity are possible, including clinically-derived values.

Based on the personal medication sensitivity, a medication sensitivity coefficient or coefficients can be found by proportioning the personal sensitivity to the population-based activity curve, if available (operation 153). The coefficient can be determined through area estimation, as further described below for dosed insulin with reference to FIG. 15. Finally, once determined, the coefficient can be applied to the population-based activity curve to generate a personal activity model for the particular antidiabetic or oral medication (operation 154).

Calibrating an Antidiabetic or Oral Medication Activity Curve

Changes to diabetes management are expected for Type 2 diabetics. However, dietary habits, personal well being, and other factors affecting both Type 1 and Type 2 diabetics can require adjustment to antidiabetic and oral medication activity curves. FIG. 14 is a process flow diagram showing a routine 160 for calibrating an antidiabetic and oral medication activity curve for use with the method 120 of FIG. 10. Calibration uses self-testing data to corroborate and refine the management tool.

Calibration can be performed regularly, or only as needed, based on factors relating to insulin, antidiabetic and oral medication, and lifestyle, as further described below with reference to FIG. 19. During each calibration, external feedback regarding the medication is aggregated into the management tool (operation 161) and applied to re-evaluate the medication sensitivity coefficient (operation 162). In addition, the feedback can be aggregated over several sample sets, or types of feedback, as further described below with reference to FIG. 20. In a further embodiment, a threshold is applied to the feedback to prevent oscillations in changes to the activity curve due to minor and insignificant fluctuations. A revised personal antidiabetic or oral medication activity model can then be generated (operation 163) as a reflection of current circumstances.

### Personalizing a Population-Based Insulin Activity Curve

Insulin is a peptide hormone composed of amino acid residues. Conventional insulin preparations are human insulin analogs that provide therapeutic effect along a projected activity curve that is characterized by time of onset, peak time, and duration of action. FIG. 15 is a graph 170 showing, by way of example, an insulin activity curve 171 for lispro, an insulin analog manufactured by Eli Lilly and Company, Indianapolis, Ind., and marketed under the Humalog brand name. The x-axis represents time in minutes and the y-axis represents the rate of glucose infusion measured in milligrams per minute per kilogram (mg/min/kg). Insulin activity curves are widely available for other insulin preparation types, and form the same general profile varied by time of onset, peak time, and duration.

The insulin activity curves published by insulin manufacturers and other authoritative sources are generally constructed as glucose clamp curves from normal volunteers, rather than diabetics, so resultant insulin activity curves must be estimated from the published curves. Insulin activity can be modeled for a diabetic patient by first estimating insulin sensitivity for an insulin preparation type. The insulin sensitivity refers to the overall change in blood glucose for a given dose of insulin, which the equivalent of integrating the area A under the insulin activity curve 171 and proportioning the area A to the net change in blood glucose 172. Thus, insulin sensitivity s can be estimated by taking the first order derivative of the rate of change of blood glucose over time:

$$s = \int \frac{d x}{d t} \qquad ( 1 )$$

where x is glucose infusion rate and t is time. Other estimates of insulin sensitivity are possible.

The insulin sensitivity is then proportioned to the population-based insulin activity curve 171 using an insulin sensitivity coefficient k for the patient. For example, if a 1.0 unit dose caused a 30 mg/dL drop in blood glucose, the area A would equal 30, and the magnitude of the values of each point along the x-axis are adjusted to the ratio of the insulin sensitivity coefficient k to the population-based value to yield a bioactivity curve for a 1.0 unit dose. Other applications of insulin sensitivity coefficients are possible.

Personal Insulin Activity Curve

The insulin sensitivity coefficient can be applied to the population-based insulin activity curve to generate a personal insulin activity model for the patient. FIG. 16 is a diagram showing, by way of example, a screen shot 180 of a personal insulin activity curve for display in the graphical user interface 80 of FIG. 6. The x-axis 181 again represents time in minutes and the y-axis 182 represents incremental blood glucose decrease measured in mg/dL.

The personal insulin activity model can be depicted through an approximation, plotted as a patient-specific insulin activity curve 183, which mimics the shape of the population-based insulin activity curve by a curvilinear ramp 184 to the peak activity time, followed by an exponential decay τ 126. A first modeling coefficient is used for the time to peak activity, called the filter length. A second coefficient is used for overall duration or decay of activity τ. The insulin sensitivity coefficient is applied to the population-based insulin activity curve through the filter length and τ. Thus, for a patient insulin sensitivity coefficient of 90%, for example, the patient-specific insulin activity curve 183 reflects a ten percent decrease in insulin sensitivity over corresponding population-based results. Other forms of and coefficients for models of population-based insulin activity curves are possible.

In a further embodiment, a time factor adjustment can be applied to get an overall insulin activity curve appropriately adjusted for an individual diabetic. For example, if the insulin activity curve for the individual was shorter in duration, the population-based insulin activity curve would be proportionally decreased along the time axis. Other personal insulin activity models are possible.

### Digestive Response Modeling

Digestive speed and amplitude are initially modeled through ingestion of a standardized test meal from which a digestive response curve can be established and calibrated. The digestive response curve is thus adapted to the patient by factoring individual sensitivities into the personal predictive management tool.

In a manner similar to insulin activity curve determination, digestive response curve establishment and calibration provides a model from which other food responses can be projected. FIG. 17 is a process flow diagram showing a routine 190 for establishing a digestive response curve for use with the method 120 of FIG. 10. Determining a digestive response curve is an empirical process performed by the patient prior to commencing automated diabetes management and repeated as necessary to calibrate or fine-tune the curve.

Initially, the patient must undertake a fast (operation 191), preferably overnight and limited to only clear liquids or water. Following fasting, an initial test for blood glucose level is made (operation 192) to establish a starting point for blood glucose rise. The patient thereafter consumes a standardized and timed test meal (operation 193), such as a specific number of oat wafers, manufactured, for instance, by Ceapro Inc., Edmonton, Canada, or similar calibrated consumable. The test meal contains a known quantity of carbohydrate. A second test for postprandial blood glucose is made after a set time period (operation 194). If desired, further post-meal blood glucose tests can be performed (not shown), although standardized test meals are designed to exhibit peak blood glucose rise after a fixed time period and further testing generally yields nominal additional information. Finally, a carbohydrate sensitivity coefficient established by plotting the observed baseline and peak blood glucose levels on a personal digestive response curve (operation 195). The protocol can be repeated, as needed, to ascertain and resolve any variability in testing results. In a further embodiment, population-based digestive response curves can be used in lieu of or in combination with an empirically-determined personal digestive response curve.

Personal Digestive Response Curve

A digestive response curve estimates digestive speed and amplitude for an individual patient, which traces blood glucose rise, peak, and fall following food consumption. FIG. 18 is a graph 200 showing, by way of example, a personal digestive response curve 203 for a standardized meal. The x-axis 201 represents time in minutes and the y-axis 202 represents a cumulative rise of blood glucose measured in milligrams per deciliter (mg/dL). The amplitude of the curve 203 is patient-dependent, as is the timing of the rise. However, where the carbohydrate content of the standardized test meal is precisely known, the curve 203 can be adapted to other types of foods to estimate glycemic effect and counteraction by insulin dosing, such as described in commonly-assigned U.S. patent application, entitled “System And Method For Actively Managing Type 1 Diabetes Mellitus On A Personalized Basis,” Ser. No. ______, pending; U.S. patent application, entitled “System and Method for Managing Type 1 Diabetes Mellitus Through a Personal Predictive Management Tool,” Ser. No. ______, pending, U.S. patent application, entitled “System And Method For Actively Managing Type 2 Diabetes Mellitus On A Personalized Basis,” Ser. No. ______, pending; U.S. patent application, entitled “System and Method for Managing Type 2 Diabetes Mellitus Through a Personal Predictive Management Tool,” Ser. No. ______, pending, the disclosures of which are incorporated by reference.

### Characteristics Affecting Management of Type 1 and Type 2 Diabetics

A range of characteristics affect the effectiveness of the automated diabetes management tool. FIG. 19 is a process flow diagram showing, by way of example, characteristics 211 affecting diabetes management 210. Other characteristics can also affect diabetes management

Type 1 diabetics are dependent on externally supplied insulin, as well as end-stage Type 2 diabetes with failed insulin production. The basic principle underlying insulin therapy is to use short-acting insulin to cover meals and longer-acting insulin between meals and overnight. Thus, insulin considerations 212 include the type of insulin preparation administered and the timing and doses of insulin. Additionally, insulin can be administered through subcutaneous injection, insulin pump, inhalation, and transdermal delivery. Other modes of insulin administration are possible. Other insulin considerations are possible.

Lifestyle considerations 213 factor heavily into determinations of insulin dosing. Food consumption considerations 214 include the types, amounts, and combinations of foods consumed, particularly in terms of carbohydrate content and glycemic index. Food includes beverages that will lead to an eventual rise in blood glucose, such as high sucrose drinks, like orange juice. In addition, personal food preferences, familial traditions, preferred seasonings and accompaniments, ethnicity, social eating patterns, and similar factors can also indirectly influence blood glucose. Exercise considerations 215 includes any form of physical exertion or activity likely to require a measurable caloric outlay. Finally, patient condition 216 can cause blood glucose to abnormally rise or fall, depending upon the condition. For instance, a virus, such as influenza, can cause blood glucose to decrease, while emotional stress can raise blood glucose through stimulation of adrenaline. The normal pancreas in non-diabetic individuals manages all of these shifts in glucose metabolism smoothly to prevent both too high and too low values of glucose. Other lifestyle considerations are possible.

Type 2 diabetics also suffer some combination of defective insulin secretion, insulin resistance, and reduced insulin sensitivity 217. The level of affect tends to change over time as the disease progresses, although, at least in the early stage, positive changes to exercise, diet, and weight loss may temporarily reverse insulin resistance. Other insulin resistance considerations are possible.

### Factors Bearing on Type 1 and Type 2 Diabetics Management

Diabetes management is a dynamic process that must evolve with patient condition to remain effective, especially for Type 2 diabetics. FIG. 20 is a process flow diagram showing, by way of example, factors 221 bearing on personal predictive diabetes management 220. Each factor, when known, constitutes feedback that can potentially have an affect on the efficacy and accuracy of an underlying diabetes management tool.

Blood glucose testing results provide strong corroboration of the management tool's accuracy. Test results can be provided through empirical measures 222 from self-testing, which can be compared to expected blood glucose levels as predicted by the management tool. Similarly, clinical monitoring data 223, such as glycated hemoglobin, fructosamine, urinary glucose, urinary ketone, and interstitial glucose testing results, can be used. Other forms of blood glucose testing results are possible.

Other factors may also apply. For instance, event data 224 details specifics, such as insulin basal dose, insulin bolus dose, insulin bolus timing, insulin resistance level, period of day, time of day, medications, patient activity level, and patient physical condition. Other forms of event data are possible. Insulin preparation type 225 should seldom vary, but when affected, can signal the need to re-evaluate and calibrate the management tool. Finally, selecting a population-based insulin activity curve for a patient population most appropriately corresponding to the quantitative characteristics 226 of the patient can improve the type of personal insulin activity model generated and subsequently refined to match the specific needs of the patient. Finally, the types and dosing of antidiabetic and oral medications 226 for Type 2 and select Type 1 diabetics can directly or indirectly affect blood glucose. Moreover, the medications taken by a Type 2 will likely change as the disease progresses. Other factors bearing on diabetes management are possible.

### System

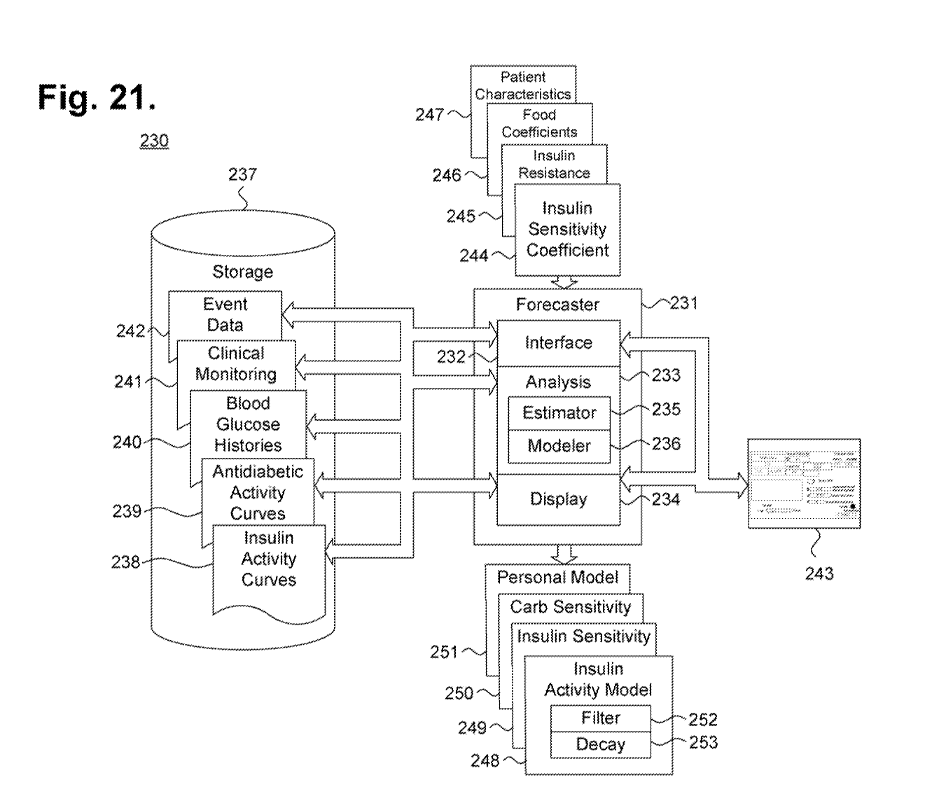

Automated diabetes management can be provided on a system implemented through a patient-operable device, as described above with reference to FIG. 5. FIG. 21 is a block diagram showing for a system 230 for establishing a personalized diabetes management tool for Type 1 and Type 2 diabetes mellitus, in accordance with one embodiment. The patient-operable device must accommodate user inputs, provide a display capability, and include local storage and external interfacing means.

In one embodiment, the system 230 is implemented as a forecaster application 231 that includes interface 232, analysis 233 and display 234 modules, plus a storage device 237. Other modules and devices are possible.

The interface module 232 accepts user inputs, such as insulin sensitivity coefficient 244, insulin resistance 245 (Type 2 only), food coefficients 246, and patient-specific characteristics 247. Other inputs, both user-originated and from other sources, are possible. In addition, in a further embodiment, the interface module 232 facilitates direct interconnection with external devices, such as a blood or interstitial glucose monitor, or centralized server (not shown). The interface module 232 can also provide wired or wireless access for communication over a private or public data network, such as the Internet. Other types of interface functionality are possible.

The analysis module 233 includes estimator 235 and modeler 236 submodules. The estimator submodule 235 determines an insulin sensitivity 249 by taking a derivative of the rate of change of blood glucose over time of a population-based insulin activity curve 158 maintained on the storage device 237. The modeler submodule 236 forms an insulin activity model 248 of the population-based insulin activity curve 158 by determining a filter length 252 and exponential decay 254. The modeler submodule 236 also forms activity curves for antidiabetic and oral medications 239, as applicable, and a carbohydrate sensitivity 250 that includes a personal digestive response curve 203 (shown in FIG. 18) determined, for instance, from a patient-supplied carbohydrate sensitivity coefficient 246 or through empirical testing with a standardized test meal. The insulin sensitivity coefficient 242 is applied to the insulin activity model 248 to form a patient-specific insulin response curve 183 (shown in FIG. 16), which is combined with the antidiabetic and oral medication activity curves 239, if applicable, and personal digestive response curve 202 to build a personalized diabetes management tool 251.

In a further embodiment, population-based digestive response curves 238, blood glucose histories 240, clinical monitoring 161, food profiles 241, and event data 242, as well as other external forms of data, are also maintained on the storage device 237. This information is used to re-evaluate the insulin sensitivity coefficient 242 and to calibrate the personal insulin activity model 247. Other types of analysis functionality are possible.

Finally, the display module 234 generates a graphical user interface 243, through which the user can interact with the forecaster 231. The user interface 243 and its functionality are described above with reference to FIG. 6.

While the invention has been particularly shown and described as referenced to the embodiments thereof, those skilled in the art will understand that the foregoing and other changes in form and detail may be made therein without departing from the spirit and scope.

What is claimed is:

1. A system for creating a personalized tool predicting a time course of blood glucose affect in diabetes mellitus, comprising:
stored information, comprising:
a substance whose introduction into a diabetic patient triggers a physiological effect relative to the diabetic patient's blood glucose; and
a time course and an amplitude of change over which the physiological effect is expected to occur;
an analysis module configured to determine a factor specific to the physiological effect on the diabetic patient and to adjust the time course and the amplitude of change in relation to the diabetic patient-specific factor; and
a modeler module configured to model the physiological effect on a curve with the time course and the amplitude of change mapped as a function of a quantity of the substance.

2. A system according to claim 1, wherein a dosed insulin preparation is specified as the substance and an insulin sensitivity is specified as the factor, such that the affect of the substance comprises mediating transport of blood glucose into cells in proportion to the insulin sensitivity.

3. A system according to claim 2, wherein the stored information further comprises an insulin activity curve for the dosed insulin preparation comprising a time course and an amplitude of change applicable to a patient population, the system further comprising:
an evaluation module configured to identify the insulin sensitivity by taking a derivative of the rate of change of blood glucose over time for the dosed insulin preparation, wherein the insulin sensitivity is applied to the patient population insulin activity curve over a duration of action of the dosed insulin preparation.

4. A system according to claim 2, wherein the diabetic patient comprises a Type 1 diabetic and the dosed insulin preparation is required for inclusion in the stored information.

5. A system according to claim 1, wherein one of an antidiabetic medication and an oral medication is specified as the substance and a physiological reaction of the patient's body to the medication is specified as the factor, such that the affect of the substance comprises triggering a hematological interaction with blood glucose as the physiological reaction.

6. A system according to claim 1, wherein carbohydrates are specified as the substance and a carbohydrate sensitivity is specified as the factor, such that the affect of the substance comprises causing a rise in blood glucose in proportion to the carbohydrate sensitivity.

7. A system according to claim 1, wherein the diabetic patient comprises a Type 2 diabetic, further comprising:
during an early stage of Type 2 diabetes, wherein the substance is omitted from the stored information;
during a middle stage of the Type 2 diabetes, wherein one of an antidiabetic medication and an oral medication is further specified as the substance and a physiological reaction of the patient's body to the medication as the factor; and
during an end-stage of the Type 2 diabetes, wherein a dosed insulin preparation is further specified as the substance and an insulin sensitivity as the factor.

8. A method for creating a personalized tool predicting a time course of blood glucose affect in diabetes mellitus, comprising:
selecting a substance whose introduction into a diabetic patient triggers a physiological effect relative to the diabetic patient's blood glucose;
determining a time course and an amplitude of change over which the physiological effect is expected to occur;
adjusting the time course and the amplitude of change in relation to a factor specific to the physiological effect on the diabetic patient; and
modeling the physiological effect on a curve with the time course and the amplitude of change mapped as a function of a quantity of the substance.

9. A method according to claim 8, further comprising:
specifying a dosed insulin preparation as the substance and an insulin sensitivity as the factor, wherein the affect of the substance comprises mediating transport of blood glucose into cells in proportion to the insulin sensitivity.

10. A method according to claim 9, further comprising:
identifying an insulin activity curve for the dosed insulin preparation comprising a time course and an amplitude of change applicable to a patient population;
determining the insulin sensitivity by taking a derivative of the rate of change of blood glucose over time for the dosed insulin preparation; and
applying the insulin sensitivity to the patient population insulin activity curve over a duration of action of the dosed insulin preparation.

11. A method according to claim 9, wherein the diabetic patient comprises a Type 1 diabetic, further comprising:
requiring inclusion of the dosed insulin preparation.

12. A method according to claim 8, further comprising:
specifying one of an antidiabetic medication and an oral medication as the substance and a physiological reaction of the patient's body to the medication as the factor, wherein the affect of the substance comprises triggering a hematological interaction with blood glucose as the physiological reaction.

13. A method according to claim 8, further comprising:
specifying carbohydrates as the substance and a carbohydrate sensitivity as the factor, wherein the affect of the substance comprises causing a rise in blood glucose in proportion to the carbohydrate sensitivity.

14. A method according to claim 8, wherein the diabetic patient comprises a Type 2 diabetic, further comprising:
during an early stage of Type 2 diabetes, omitting the substance;
during a middle stage of the Type 2 diabetes, further specifying one of an antidiabetic medication and an oral medication as the substance and a physiological reaction of the patient's body to the medication as the factor; and
during an end-stage of the Type 2 diabetes, further specifying a dosed insulin preparation as the substance and an insulin sensitivity as the factor.

15. A system for establishing a tool of blood glucose change for diabetes mellitus management in an individual patient, comprising:
a database comprising factors specific to a diabetic patient, comprising:
an insulin sensitivity for an insulin preparation for treatment of diabetes mellitus; and
a carbohydrate sensitivity for a known quantity of carbohydrate, which is measured postprandial after a fixed time period;
a modeler module configured to build a management tool for the diabetic patient, comprising:
a time course for a dose of the insulin preparation with an amplitude of change proportioned to the insulin sensitivity; and
a time course for an amount of carbohydrate with an amplitude of change proportioned to the carbohydrate sensitivity; and
a calibration module configured to calibrate the management tool by aggregating feedback from testing of blood glucose into at least one of the insulin and the carbohydrate sensitivities.

16. A system according to claim 15, wherein the database further comprises a population-based time course for a dose of the insulin preparation with an amplitude of change proportioned for a test patient population, the system further comprising:
an analysis module configured to apply the insulin sensitivity for a quantity comparable to the insulin preparation dose to the population-based time course.

17. A system according to claim 15, wherein the insulin sensitivity s is estimated by taking a first order derivative of a rate of change of blood glucose over time in accordance with:
$$s = \int \frac{d x}{d t}$$
where x is glucose infusion rate and t is time.

18. A system according to claim 15, wherein the carbohydrate sensitivity is determined through ingestion of a standardized test meal comprising the known quantity of carbohydrate, the system further comprising:
a monitoring module configured to observe a baseline blood glucose and a peak blood glucose prior and postprandial to the standardized test meal; and
an analysis module configured to evaluate the baseline and the peak blood glucose levels to establish the carbohydrate sensitivity.

19. A system according to claim 15, further comprising:
a prediction module configured to forecast the blood glucose by application of the management tool predicated on at least one of a dose of the insulin preparation and ingestion of an amount of carbohydrate, wherein the blood glucose of the diabetic patient is observed as the feedback following actually taking the dose or postprandial to the ingestion.

20. A system according to claim 15, wherein the blood glucose is measured through one of extracorporeal and interstitial glucose testing

21. A method for establishing a tool of blood glucose change for diabetes mellitus management in an individual patient, comprising:
determining factors specific to a diabetic patient, comprising:
identifying an insulin sensitivity for an insulin preparation for treatment of diabetes mellitus; and
identifying a carbohydrate sensitivity for a known quantity of carbohydrate, which is measured postprandial after a fixed time period;
generating a management tool for the diabetic patient, comprising:
mapping, a time course for a dose of the insulin preparation with an amplitude of change proportioned to the insulin sensitivity; and
mapping a time course for an amount of carbohydrate with an amplitude of change proportioned to the carbohydrate sensitivity; and
calibrating the management tool by aggregating, feedback from testing, of blood glucose into at least one of the insulin and the carbohydrate sensitivities.

22. A method according to claim 21, further comprising:
referencing a population-based time course for a dose of the insulin preparation with an amplitude of change proportioned for a test patient population; and
applying the insulin sensitivity for a quantity comparable to the insulin preparation dose to the population-based time course.

23. A method according to claim 21, further comprising:
estimating the insulin sensitivity s by taking a first order derivative of a rate of change of blood glucose over time in accordance with:
$$s = \int \frac{d x}{d t}$$
where x is glucose infusion rate and t is time.

24. A method according to claim 21, further comprising:
determining the carbohydrate sensitivity through ingestion of a standardized test meal comprising the known quantity of carbohydrate;
observing a baseline blood glucose and a peak blood glucose prior and postprandial to the standardized test meal; and
evaluating the baseline and the peak blood glucose levels to establish the carbohydrate sensitivity.

25. A method according to claim 21, further comprising:
forecasting the blood glucose by application of the management tool predicated on at least one of a dose of the insulin preparation and ingestion of an amount of carbohydrate; and
observing the blood glucose of the diabetic patient as the feedback following actually taking the dose or postprandial to the ingestion.

26. A method according to claim 21, further comprising:
measuring the blood glucose through one of extracorporeal and interstitial glucose testing.
